(19) United States
(12) Patent Application Publication
(10) Pub. No.: US 2024/0192230 A1
(43) Pub. Date: Jun. 13, 2024

(54) Digital Protein Misfolding Assays

(71) Applicant: The Brigham and Women’s Hospital, Inc., Boston, MA (US)

(72) Inventors: David R. Walt, Boston, MA (US); Maia Norman, Boston, MA (US); Zoe Swank, Boston, MA (US); Tal Gilboa, Boston, MA (US)

(21) Appl. No.: 18/286,903

(22) PCT Filed: Apr. 15, 2022
(86) PCT No.: PCT/US2022/071746

§ 371 Date: Oct. 13, 2023

Related U.S. Application Data
(60) Provisional application No. 63/313,229, filed on Feb. 23, 2022.
(60) Provisional application No. 63/175,503, filed on Apr. 15, 2021.

(51) Int. Cl. G01N 33/68; G01N 33/533

(52) U.S. Cl. CPC G01N 33/6896; G01N 33/533; G01N 33/6854; G01N 2333/4709; G01N 2800/28

(57) ABSTRACT

Described herein are quantitative methods that can be used for diagnosis of neurodegenerative diseases, characterized partially by pathological protein aggregation and/or deposition, comprised of assaying in microwells, droplets, beads or some other support, the ability of a pathological protein in a biological sample to seed the conversion of substrate monomers into aggregated fibrils.

### CLAIM OF PRIORITY

This application claims the benefit of U.S. Provisional Application Ser. No. 63/175,503, filed on Apr. 15, 2021, and 63/313,229, filed on Feb. 23, 2022. The entire contents of the foregoing are incorporated herein by reference.

### TECHNICAL FIELD

Described herein are quantitative methods that can be used for diagnosis of neurodegenerative diseases, characterized partially by disease-associated protein aggregation and/or deposition, comprised of assaying in microwells, droplets, beads or some other support, the ability of a pathological protein in a biological sample to seed the conversion of substrate monomers of disease-associated proteins into aggregated fibrils.

### BACKGROUND

Parkinson's disease (PD) is a devastating neurological movement disorder for which there are currently no approved diagnostics and few effective therapies. Currently, the second most common neurodegenerative disease in the United States, PD affects over one million adults.¹

### SUMMARY

Provided herein are methods for detecting, and optionally quantifying a level of, pathological protein aggregates (e.g., α-synuclein, PrP, amyloid beta, TDP-43, and/or tau aggregates or pathological seeds thereof) in a sample from a subject; in some embodiments the sample comprises a biofluid, e.g., cerebrospinal fluid, plasma, or serum, or extracellular vesicles derived therefrom. The methods can comprise providing a sample from a subject, optionally a subject who is known or suspected to have a condition associated with aggregation of disease-associated proteins; optionally enriching the sample for extracellular vesicles; diluting a portion of the sample by addition of a diluent comprising reaction reagents to form a reaction mixture, preferably wherein the reaction reagents comprise monomers of disease-associated proteins and optionally one or more detection reagents; compartmentalizing the reaction mixture into separate confined volumes, preferably wherein each of the confined volumes has a volume of 1 pL to 1000 nL, preferably 10 pL to 1000 nL, 100 pL to 500 nL, 1 to 500 nL, 10 to 500 nL, 0.1 to 100 nL, 1 to 100 nL, 10 to 100 nL, or 10 pL to 1 nL, and wherein each confined volume has zero or one pathological protein aggregate; incubating the confined volumes under conditions to allow co-aggregation of the monomers of disease-associated protein and any pathological protein aggregates present in the sample; detecting the presence or absence of co-aggregates of the disease-associated protein monomers and pathological protein aggregates in each confined volume, wherein the formation of co-aggregates indicates the presence of pathological protein aggregates in the confined volume; and optionally quantifying the level of pathological aggregates in the sample by counting the number of confined volumes comprising co-aggregates.

Also provided herein are multiplexed methods for detecting, and optionally quantifying a level of, pathological protein aggregates of two or more different disease-associated proteins (e.g., α-synuclein, PrP, amyloid beta, TDP-43, and/or tau aggregates or pathological seeds thereof) in a sample from a subject, preferably wherein the sample comprises cerebrospinal fluid, plasma, or serum. The methods can comprise providing a sample from a subject, optionally a subject who is known or suspected to have a condition associated with aggregation of disease-associated proteins; optionally enriching the sample for extracellular vesicles; mixing the sample with a plurality of fluorescently labeled beads, wherein the beads are coated with antibodies that bind specifically to each of the two or more co-aggregates, preferably α-synuclein, PrP, amyloid beta, TDP-43, and/or tau co-aggregates, wherein each fluorescent label is associated with antibodies that bind specifically to a specific protein aggregate; diluting the mixture of sample and beads by addition of a diluent comprising reaction reagents to form a reaction mixture, preferably wherein the reaction reagents comprise monomers of disease-associated proteins and a detection reagent; compartmentalizing the reaction mixture into separate confined volumes, preferably wherein each of the confined volumes has a volume of 1 pL to 1000 nL, preferably 10 pL to 1000 nL, 100 pL to 500 nL, 1 to 500 nL, 10 to 500 nL, 0.1 to 100 nL, 1 to 100 nL, 10 to 100 nL, or 10 pL to 1 nL, and wherein each confined volume has zero or one pathological protein aggregate; incubating the confined volumes under conditions to allow co-aggregation of the monomers of disease-associated protein and any pathological protein aggregates present in the sample; detecting the presence or absence of co-aggregates of the disease-associated protein monomers and pathological protein aggregates in each confined volume, wherein the formation of co-aggregates indicates the presence of pathological protein aggregates in the confined volume; determining the identity of the co-aggregates based on the identity of fluorescent labels, and optionally quantifying the level of each of the pathological protein aggregates in the sample by counting the number of confined volumes comprising co-aggregates.

In some embodiments, the sample is diluted to a 1:1 to 1:100 ratio of sample:diluent. For example, the dilution can be 1:1, 1:2, 1:5, 1:10, 1:25, 1:50, 1:75, 1:80, or 1:100, with each of these forming an upper or lower limit of a range of dilutions.

In some embodiments, compartmentalizing the reaction mixture into separate confined volumes comprises partitioning aliquots of the reaction mixture into droplets, microwells, or hydrogel microspheres. In some embodiments, the sample is diluted before being compartmentalized; alternatively, the sample can be added to diluent already present in the confined volume compartments (e.g., microwells) to reach the final dilution.

In some embodiments, the methods further comprise heating the confined volumes to 35-50° C. prior to detecting the presence or absence of co-aggregates.

In some embodiments, the methods further comprise mixing the sample with beads prior to compartmentalization and incubation. In some embodiments, the beads are coated with antibodies that bind specifically to the co-aggregates.

In some embodiments, detecting the presence or absence of co-aggregates comprises detecting the presence or absence of a detection reagent signal.

In some embodiments, the detection reagent is Thioflavin T (ThT) or X-34, and detecting the presence or absence of a protein aggregate comprises detecting a fluorescence signal from the detection reagent.

In some embodiments, the condition associated with aggregation of pathological proteins and the pathological protein monomers are as shown in Table A.

In some embodiments, the condition associated with aggregation of pathological proteins is Parkinson's disease (PD), and the pathological protein monomers comprise α-synuclein. In some embodiments, the condition associated with aggregation of pathological proteins is transmissible spongiform encephalopathies (TSEs) and the pathological protein monomers comprise prion protein (PrP). In some embodiments, the condition associated with aggregation of pathological proteins is Alzheimer's disease (AD), and the pathological protein monomers comprise Amyloid Beta. In some embodiments, the condition associated with aggregation of pathological proteins is amyotrophic lateral sclerosis (ALS) and frontotemporal lobar degeneration (FTD), and the pathological protein monomers comprise transactive response DNA-binding protein 43 (TDP-43). In some embodiments, the condition associated with aggregation of pathological proteins is Pick disease, Alzheimer's disease (AD), chronic traumatic encephalopathy (CTE), progressive supranuclear palsy (PSP), corticobasal degeneration (CBD), frontotemporal dementias associated with MAPT mutations (FTDP 17 MAPT), and the pathological protein monomers comprise Tau.

In some embodiments, the methods further comprise identifying the subject as having a condition associated with aggregation of pathological proteins, e.g., as shown in Table A. Optionally, the methods can include recommending, suggesting, and/or administering a treatment, e.g., as shown in Table B.

Also provided herein are quantitative methods for the diagnosis (e.g., using RT-QuiC) of neurodegenerative diseases, characterized partially by pathological protein aggregation and/or deposition (of disease-associated proteins), comprised of assaying in microwells, droplets, beads (e.g., on antibody-coated magnetic beads) or some other support, or some other compartment, the ability of a pathological protein in a biological sample to seed the conversion of substrate monomers into aggregated fibrils thereby measuring said fibril formation using an aggregation-sensitive dye to visualize said formation (a digital seed amplification assay, e.g., RT-QuiC) wherein: said pathological protein in the biological sample is quantified by diluting said biological sample so that the aggregate concentration in each microwell (or other support or compartment) is either 0 or 1, thus keeping within Poisson statistics, and then assaying the formation of aggregated fibrils over time.

In some embodiments, the biological sample is a fluid or vesicle. In some embodiments, the fluid or vesicle includes, but is not limited to, (a) plasma; (b) serum; (c) cerebrospinal fluid; (d) urine; (e) exosomes; or (f) extracellular vesicles. In some embodiments, the dye is Thioflavin T or any other aggregation-sensitive dye. In some embodiments, the aggregation-sensitive dye can be used as, but is not limited to, an enzymatic label, a fluorescent label, a radioactive label, or a metal label. In some embodiments, the enzymatic label is selected from the group consisting of beta-galactosidase, horseradish peroxidase, glucose oxidase, and alkaline phosphatase.

In some embodiments, the substrate support surface may include, but not limited to, beads (a generic term used interchangeably with particle or microsphere as a small discrete entity), nanotubes, polymers plates, disks or dipsticks.

In some embodiments, the type of bead may be characterized as, but is not limited to, magnetic beads, plastic beads, ceramic beads, glass beads, polystyrene beads, methylstyrene beads, acrylic polymer beads, carbon graphited beads, titanium dioxide beads, latex or cross linked dextrans such as SEPHAROSE beads, cellulose beads, nylon beads, cross-linked micelles, or TEFLON® beads.

In some embodiments, the disease-associated protein monomers, in said biological sample, includes, but is not limited to, alpha-Synuclein, Prion protein (PrPSc), Tau, Amyloid Beta (ABeta) or TDP-43.

In some embodiments, two or more (multiplex) orthogonal monomer substrates are used to detect multiple neurodegenerative disease-causing agents in a single biological sample.

In some embodiments, the quantitative assay is further used as a biomarker to indicate neurodegenerative disease progression or stage, or to assess response to treatment.

In some embodiments, the neurodegenerative disease includes, but is not limited, Parkinson's Disease (PD), Lewy Body Dementia (LBD), Multiple Systems Atrophy (MSA), Frontotemporal Dementia (FTD), Alzheimer's Disease (AD), Progressive Supranuclear Palsy (PSP), Pick's Disease (PiD), Amyotrophic Lateral Sclerosis (ALS) or Transmissible Spongiform Encephalopathy.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. A schematic illustration of an exemplary α-synuclein seed amplification assay (SAA). The SAA reaction combines pathological α-synuclein seeds, α-synuclein monomers, an amyloid fibril staining dye, and salts. After heating and shaking the reaction, pathological seeds serve as the template for generating amyloid fibrils, which can be visualized with the staining dye.

FIGS. 2A-D. Exemplary α-synuclein digital SAA (dSAA) performed in microwells (2A-B) or droplets (2C-D). A) dSAAs were performed in QUANTSTUDIO Real-Time PCR chips with and without pre-formed α-synuclein fibrils, + and − aggregates, respectively. Representative images, covering a subsection of the chip that includes approximately 80 microwells, are shown for timepoints equal to 0, 24, and 48 hours for both the positive and negative controls. The reaction solution contained 100 mM PIPES pH 6.5, 100 mM GdmCl, 0.25 mg/mL K23Q α-synuclein monomers, and 1 μM X-34 dye. The positive control reaction was supplemented with 5 ng/mL pre-formed α-synuclein fibrils. Both chips were incubated at 40° C. B) Serial dilution of pre-formed α-synuclein fibrils imaged after 92 hours. C) α-synuclein dSAA in single emulsion droplets. A fraction of the droplets were imaged at timepoints equal to 0, 24, and 96 hours. The reaction solution contained 100 mM PIPES pH 6.5, 100 mM GdmCl, 0.1 mg/mL K23Q α-synuclein monomers, 1 μM X-34 dye and 5 ng/mL pre-formed α-synuclein fibrils. D) α-synuclein dSAA in double emulsion droplets. dSAAs were performed in double emulsion droplets with and without pre-formed α-synuclein fibrils, + and − aggregates, respectively. The reaction solution contained 100 mM PIPES pH 6.5, 100 mM GdmCl, 0.1 mg/mL K23Q α-synuclein monomers and 1 μM X-34 dye. The positive control reaction was supplemented with 5 ng/ml pre-formed α-synuclein fibrils.

FIG. 3. Impact of modulating pH and ionic composition on α-synuclein aggregation. Images of aggregates formed from pre-formed α-synuclein fibrils for various solution conditions captured after 48 hours of incubation at 40° C. in QUANTSTUDIO Real-Time PCR chips. The first two images exemplify aggregates formed in a reaction using PIPES buffer at a pH of 6.5 versus 5.8. The remaining three images represent aggregates formed using three different salts: NaCl, GdmCl, and MgSO₄, and PIPES buffer at a pH of 6.5. In each case 5 ng/ml of pre-formed α-synuclein fibrils, 0.25 mg/mL of K23Q α-synuclein monomers, and 1 μM of X-34 dye were added to the reaction mixture.

FIGS. 4A-B. Bead-based dSAA in microwells. A) Schematic illustration of a bead-based dSAA. B) 0.1 ng/mL pre-formed α-synuclein fibrils were captured on antibody-coated magnetic beads, resuspended in a solution containing 100 mM PIPES pH 6.5, 100 mM GdmCl, 0.25 mg/mL K23Q α-synuclein monomers, and 1 μM X-34 dye. The beads were loaded into a microwell array and filament growth was imaged over time.

FIGS. 5A-C. Proteinase protection assays and alpha-synuclein in plasma-derived EVs. A) schematic illustration of the protection assay: Isolated EVs are treated with Proteinase K (PK) and PK inhibitor (PMSF) to quantify internal and external protein levels. B) To validate the protection assay, Alix (left graph) and Albumin (right graph) concentrations of EVs isolated from plasma were measured by Simoa for three conditions: i. No treatment, two-hour Project Proposal Template 5 incubation, lyse with Triton X, one-hour incubation; ii. PK treatment, two-hour incubation, lyse with Triton X, one-hour incubation; iii. PK treatment, one-hour incubation, PMSF treatment, one hour incubation, lyse with Triton X, one-hour incubation. C, the concentration of alpha-synuclein in EVs isolated from plasma as measured by Simoa for the three conditions, showing the presence of alpha-synuclein inside EVs.

### DETAILED DESCRIPTION

Clinicians rely on qualitative methods to diagnose Parkinson's disease (PD), making it challenging to monitor disease progression or evaluate the effect of new treatments. PD severity is strongly correlated with the accumulation of intraneuronal protein aggregates made up of α-synuclein.² Pathological forms of α-synuclein can be detected in the biofluids of PD patients using seed amplification assays (SAAs); however, in their present format, these assays are not quantitative and suffer from a lack of reproducibility.³ Described herein is a digital α-synuclein SAA to measure the concentration of pathological aggregates in patient samples, preferably samples collected with minimally invasive procedures. A quantitative SAA would likely yield new insights into the evolution of PD and can be used as a companion diagnostic in clinical trials targeting α-synuclein aggregation.

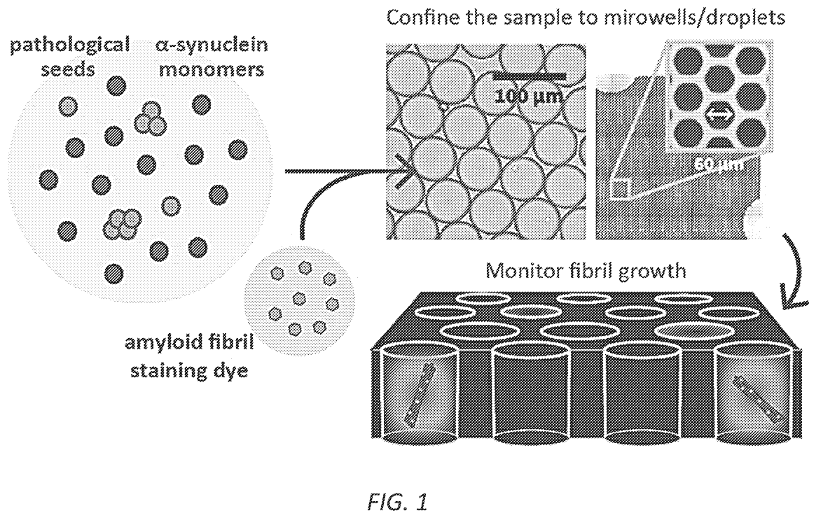

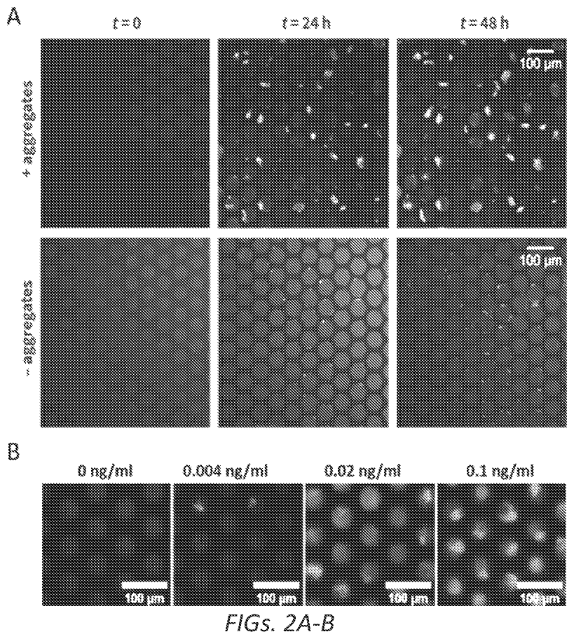

SAAs, such as real-time quaking-induced conversion (RT-QuiC) (also known as Protein Misfolding Cyclic Amplification (PMCA)), offer a promising strategy for detecting pathological protein seeds in cerebrospinal fluid (CSF).⁴ Originally developed to detect Creutzfeldt-Jakob disease by amplifying the pathological protein PrP1 (Zhu and Aguzzi, J Cell Sci (2021) 134 (17): jcs245605), the RT-QuiC assay has since been re-engineered for the diagnosis of PD based on the detection of misfolded α-synuclein,⁵⁻⁷ as well as for other diseases (Fairfoul et al., 2016. Ann. Clin. Transl. Neurol. 3, 812-818; Kraus et al., 2019. Acta Neuropathol. 137, 585-598; Rossi et al., 2020. EMBO J. 20, 694-702; Saijo et al., 2017. Acta Neuropathol. 133, 751-765)). In some of these protocols, a CSF sample is incubated with free disease-associated protein monomers, e.g., α-synuclein monomers, and Thioflavin T (ThT) and shaken (see, e.g., FIG. 1). If the relevant pathological protein seeds are present in the sample, they serve as seeds onto which free monomers of the disease-associated protein can bind, forming aggregates. The ThT in solution will bind and change conformation when it encounters an aggregate, leading to a fluorescent signal.⁸ Bulk RT-QuiC assays give an all-or-nothing signal, providing no quantitative information regarding the number of pathological protein seeds in a CSF sample. Furthermore, the assay sensitivity is limited by the number of aggregates necessary to initiate exponential growth above that of spontaneous aggregation, which can occur with only monomers present, leading to false positives. By digitizing this signal, the present methods can achieve considerably lower limits of detection by limiting spontaneous monomeric aggregation, enabling smaller quantities of aggregates to be detected. Improving sensitivity is critical because neurodegeneration is a progressive disease associated with increased cell death overtime, therefore detecting smaller quantities of aggregates at earlier timepoints may lead to better clinical outcomes.

Once PD symptoms occur, 60% of dopaminergic neurons have died, and 80% of striatal dopamine has been depleted.¹¹ Without a reliable diagnostic test for PD, a clinical diagnosis can only be made upon the presentation of motor symptoms. In addition, several neurodegenerative diseases resemble PD in its early stages, making it difficult to distinguish PD from other conditions, including multiple system atrophy (MSA), progressive supranuclear palsy (PSP), and corticobasal degeneration (CBD).¹² Despite the availability of clinical guidelines, under-diagnosis and misdiagnosis are not uncommon. The probability of misdiagnosis correlates with the clinician's specialty, where an error rate of approximately 25% occurs when the diagnosis is performed in a primary care setting compared to 6-8% for movement disorder specialists.¹³ Currently available PD medications are often most effective in the early stages of the disease, causing delayed or missed diagnoses to have deleterious effects.¹⁴ Therefore, one of the major intermediate goals for curing PD is finding effective methods for diagnosing the disease before symptom onset, allowing clinicians to track disease progression and implement preventative strategies.

Described herein are digitized SAAs, e.g., α-synuclein SAA, which allow the number of pathological aggregates or ‘seeds’ to be directly counted in a patient sample; in some embodiments, the seeds are not yet aggregated, but are misfolded and provide a scaffold upon which pathological aggregation occurs. To accomplish this digitization, small quantities of a dilute biofluid sample are confined to individual compartments, e.g., to individual microwells, droplets, or microcapsules; the sample is mixed with a diluent and reagents for the reaction to form a reaction mixture. The initial volume of the biofluid sample can be, e.g., 1 to 100 μL, 5 to 50 μL, 5 to 25 μL or 10 to 30 μL. The volume of the reaction mixture in the individual compartments can be, e.g., 1 pL to 1000 nL, preferably 10 pL to 1000 nL, 100 pL to 500 nL, 1 to 500 nL, 10 to 500 nL, 0.1 to 100 nL, 1 to 100 nL, 10 to 100 nL, or 10 pL to 1 nL; for example, the lower end of the range of volumes can be, e.g., 1 pL, 10 pL, 100 pL, 1 nL, or 10 nL, and the upper end can be, e.g., 1 nL, 10 nL, 100 nL, or 1000 nL. By diluting the biofluid, the number of aggregates in each compartment is limited to either 0 or 1, keeping within Poisson statistics. Exemplary microwell arrays are described, e.g., in Rondelez et al., Nat. Biotechnol. 2005, 23 (3), 361-365; Rissin et al., Nano Lett. 2006, 6 (3), 520-523; Cohen and Walt, Annu. Rev. Anal. Chem. 2017, 10 (1), 345-363); exemplary microfluidic droplets are described, e.g., in Kim et al., Lab. Chip 2012, 12 (23), 4986; Witters et al., Lab. Chip 2013, 13 (11), 2047; Yelleswarapu et al., Proc. Natl. Acad. Sci. 2019, 116 (10), 4489-4495; Cohen et al., ACS Nano 2020, 14, 8, 9491-9501). The aggregation reaction is run with monomers and a detection reagent, e.g., a dye, for detection of the aggregates, and the number of wells in which fluorescence appears over time (e.g., 1 to 10 days, e.g., at least 1, 2, 3, 4 or 5 days, up to 4, 5, 6, 7, 8, 9, 10 days) (positives) is counted. When the number of positives exceeds a reference number (optionally zero), the subject is identified as being at risk of developing the disease, or as having the disease; higher numbers of positives can indicate more severe or more advanced disease. In some embodiments, the methods include heating the samples to 30-60° C., e.g., 35-50° ° C., or 35-45° C. Ultimately, the number of pathological proteins in the biofluid can be quantified instead of only determining if they are present or absent.

Samples that can be used in the present methods include any biofluid, e.g., CSF, plasma, or serum. In some embodiments, the sample comprises or is enriched for extracellular vesicles (EVs), e.g., plasma-derived extracellular vesicles (EVs). α-synuclein is present inside plasma-derived EVs, as demonstrated herein using proteinase protection assays and the Simoa platform. The digital SAAs described herein can be used in other biofluids, such as blood (e.g., plasma or serum), that can be routinely obtained less invasively than CSF. In some embodiments, the assays are carried out using plasma-derived extracellular vesicles (EVs). The transport of α-synuclein aggregates from cell to cell in the brain is known to occur in EVs, and it is thought that the EV-mediated spread of α-synuclein oligomers is a prion-like process that underlies the disease.¹⁰ Therefore, EVs are likely to be a rich source of seeds for a digital SAA.

Methods for enriching the sample for (e.g., isolating) plasma-derived EVs are known in the art, and include size exclusion chromatography, immunoisolation, ultracentrifugation, PEG precipitation, and density gradient centrifugation; see, e.g., Norman et al., Nat Methods. 2021; 18(6):631-634; Ter-Ovanesyan et al., eLife. 2021; 10:e70725.

To burst the EVs and release the analytes into the sample, the methods can include the use of surfactants such as triton X (e.g., 0.01-5% triton X), Tween 20, as well as physical methods such as sonication.

Sample dilution can be, e.g., a 1:1 to 1:100 ratio of sample:diluent, e.g., wherein the diluent comprises buffer and reagents for the reaction, to form a reaction mixture; for example, the dilution can be 1:1, 1:2, 1:5, 1:10, 1:25, 1:50, 1:75, 1:80, or 1:100, with each of these forming an upper or lower limit of a range of dilutions. Suitable buffers preferably comprise salts, e.g., sodium chloride (NaCl), sodium iodide (NaI), magnesium sulfate (MgSO4), and guanidinium chloride (GdmCl), calcium chloride (CaCl₂)), and/or magnesium chloride (MgCl2). Exemplary buffers include PIPES, TRIS, or MES buffer with about 10-500 mM salts. The diluent preferably has a pH of about 5.5-7.5, e.g., about pH 6.5. A detergent can also be included, e.g., triton X (e.g., 0.01-5% triton X), Tween 20, present at about 0.01%-1% volume. The diluent can also include bovine serum albumin (BSA), crowding agents such as PEG that can accelerate the aggregation speed, and optionally lipids that interact with the pathological aggregates, e.g., alpha synuclein aggregates, and facilitate the aggregation. In some embodiments, a plurality of serial dilutions are used, e.g., comprising two or more of 1:1, 1:2, 1:4, 1:5, 1:6, 1:8, 1:10, 1:12; 1:15; 1:20; 1:25; 1:30; 1:50; 1:75, and/or 1:100, to reach a dilution that provides one aggregate per sample.

Monomers (e.g., recombinant human monomeric proteins) and a detection reagent are also added to the sample to form the reaction mixture. Detection reagents includes dyes that bind specifically to protein aggregates. Table A below provides the monomers and detector dye for each disease-associated protein.

TABLE A
Exemplary Disease-Associated Proteins

| Proteinopathy | Disease-Associated |
|---|---|
| Disease | Protein |
| Parkinson's disease (PD) | α-synuclein |
| transmissible spongiform encephalopathies | prion protein (PrP) |
| (TSEs), Creutzfeldt-Jakob disease (CJD) | |
| Alzheimer's disease (AD) | Amyloid Beta |
| Pick disease, Alzheimer disease, chronic | Tau |
| traumatic encephalopathy (CTE), progressive | |
| supranuclear palsy (PSP), corticobasal | |
| degeneration (CBD), frontotemporal | |
| dementias associated with MAPT mutations | |
| (FTDP 17 MAPT) | |
| amyotrophic lateral sclerosis (ALS) and | transactive response DNA-binding |
| frontotemporal lobar degeneration (FTD) | protein 43 (TDP-43) |

For example, an exemplary human monomneric α-syn protein monomer can comprise RefSeq ID No. NP_000336.1; see, e.g., Bargar et al., Acta Neuropathologica Communications volume 9, Article number: 62 (2021). For TDP-43, either the full length (e.g., human TDP-43 (HuTDP-43(1-414)) or a truncated form (e.g., HuTDP-43(263-414)) can be used (see, e.g., Scialò et al., Brain Commun. 2020 Sep. 14; 2(2):fcaa142). For detecting aggregation of tau, full length versions including 3 microtubule binding-repeat (3R), 4-repeat (4R), or both 3R and 4R (3R/4R) tau isoforms, as well as fragments such as K12CFh, K18CFh, K19CFh, or 1306-378 can be used (see, e.g., Metrick et al., Acta Neuropathol Commun. 2020 Feb. 22; 8(1):22; Tennant et al., Prion. 2020 December; 14(1):249-256). To detect prion protein (PrP) aggregates, recombinant PrP (rPrP) can be used (see, e.g., Green et al., Pract Neurol. 2019 February; 19(1): 49-55; Atarashi et al., Prion. 2011 July-September; 5(3): 150-153).

The bank vole protein can also be used as a substrate for detection of prion strains, see Orrú et al., PLOS Pathog. 2015 Jun. 18; 11(6):e1004983.

Detection reagents for prion proteins and amyloid beta can include Thioflavin T (ThT); azo dyes Chrysamine-G, Congo Red dye (CR) and its derivative X-34 (Styren et al., J Histochem Cytochem. 2000 September; 48(9):1223-32); crystal violet or Sirius-red staining (Cooper, J Clin Pathol. 1969 July; 22(4): 410-413); luminescent conjugated polythiophenes (LCPs), e.g., HS-84, HS-42, HS-72, polythiophene acetic acid (PTAA), p-FTAA, p-FTAA-ph, p-KTAA, and h-FTAA (Magnusson et al., Prion. 2014 July-August; 8(4): 319-329; Simon et al., Chemistry. 2014 Sep. 22; 20(39): 12537-12543); and camelid antibody domain B10 (amyloid). N,O-Benzamide difluoroboron complexes can also be used to detect β-amyloid and tau fibrils (Chen et al., Chem Commun (Camb). 2020 Jul. 2; 56(53):7269-7272); fluorescence imaging or detection can be used to detect formation of aggregates. In preferred embodiments, ThT fluorescence (450 nm excitation and 480 nm emission) or X-34 (367 nm excitation and 497 nm emission) is used.

### Beads and Microcapsules

In some embodiments, the methods include pre-capturing the aggregates with micro- and/or nanoparticles (e.g., microbeads) that are coated with antibodies that bind to the aggregates (e.g., antibodies specific for the aggregated conformation, e.g., of α-synuclein). This method can generally include two steps: in the first step the beads are mixed with the diluted biofluid. Preferably an excess of beads is used (e.g., 100,000 to 1,000,000 beads) such that each bead will capture either 0 or 1 aggregate, keeping within Poisson statistics. In the second step the beads are washed, resuspended in the working buffer and are loaded into the compartments for the SAA.

For example, a patient biofluid can be incubated with multiple fluorescently labeled beads, each coated with an antibody specific to a given protein aggregate, e.g. α-synuclein, PrP, Tau or ABeta, such that a particular fluorophore is associated with each of the protein aggregates. The beads can then be resuspended in the SAA reaction mixture, containing all the protein monomers associated with the aggregates targeted for detection, and loaded into microwell arrays or encapsulted into droplets. The amyloid staining dye is non-discriminant and therefore binds any growing aggregate. Rather, the type of protein aggregate is determined according to the fluorescent labeled bead, which is coated with an antibody specific to a given protein aggregate.

Antibodies specific to a given protein aggregate, e.g., α-synuclein, PrP, Tau, or ABeta, are known, as are methods for making such antibodies; see, e.g., Tayebi et al., PLOS One. 2011; 6(5):e19998 (PRIOC mAbs, PrP(Sc)-specific antibodies that detect PrPSc oligomers/multimers); Kumar et al., Neurobiol Dis. 2020 December; 146:105086 (α-synuclein conformation-specific antibodies); Gibbons et al., Mol Neurodegener. 2020 Nov. 4; 15(1):64 (conformation-selective tau monoclonal antibodies DMR7 and SKT82); Esteves-Villanueva et al., Biochemistry. 2015 Jan. 20; 54(2):293-302 (anti-tau aggregate antibodies including anti-tau 259-266 (“Paired-262”), anti-tau 341-360 (“A-10”)); Rofo et al., Translational Neurodegeneration volume 10, Article number: 38 (2021) (antibodies that bind to AB, including mAb158, Hexa-RmAb158, aducanumab, and lecanemab (BAN2401)); and Gibbs et al., Scientific Reports volume 9, Article number: 9870 (2019) (PMN310, which binds to amyloid-beta oligomers (ABO)).

The micro- and/or nanoparticles (e.g., microbeads) can be made of various materials. In general, any polymeric or plastic materials can be used to create the microparticles, microbeads, or nanoparticles, including materials such as polystyrene and polyethylene, for example. In some embodiments, microparticles can be formed of biologically-compatible polymer materials such as polyacrylates, polymethacrylates, and/or polyamides.

In certain embodiments, metallic, metal-oxide, semiconductor, and/or semiconductoroxide micro- and/or nanoparticles formed from one or more of Au, Ag, Pt, Al, Cu, Ni, Fe, Cd, Se, Ge, Pd, Sn, iron oxide, TiO₂, Al₂O₃, and SiO₂ can be made in many sizes and used. For example, monocrystalline iron oxide nanoparticles (MIONs) and crosslinked iron oxide (CLIO) particles can be used. In some embodiments, the beads are paramagnetic. Suitable beads include, but are not limited to, magnetic beads (e.g., paramagnetic beads), plastic beads, ceramic beads, glass beads, silica beads, polystyrene beads, methylstyrene beads, acrylic polymer beads, carbon graphited beads, titanium dioxide beads, latex or cross-linked dextrans such as SEPHAROSE beads, cellulose beads, nylon beads, cross-linked micelles, and TEFLON® beads. In some embodiments, spherical beads are used, but non-spherical or irregularly-shaped beads may be used.

In some embodiments, the beads are as described in U.S. Pat. No. 8,222,047, 8,460,879, 8,492,098, 8,846,415, 9,310,360, 9,395,359, or 9,482,662; or WO2020037130.

In some embodiments, the methods includes encapsulating aggregates in permeable compartments that permit the exchange of reagents during the SAA reaction. For example, hydrogel microcapsules that are made with an alginate hydrogel shell have a pore size of 10-20 nm, e.g., about 16 nm,⁴³ would allow the diffusion of fresh monomers, dye, and salt into the reaction vessel, while the aggregate bound bead would remain inside. It would then be possible to continually replenish reagents, increasing the amplification of aggregates and potentially improving the signal to background ratio. See, e.g., US20210263049, WO2021262706, Huang et al., Lab Chip. 2017 May 31; 17(11): 1913-1932; Di Girolamo et al., Microbial Cell Factories volume 19, Article number: 170 (2020); Mazutis et al., Macromol Biosci. 2015; 15(12):1641-1646.

### Assays

General RT-quic assay methods are described, e.g., in US20090047696, US20130288389, Wilham et al., PLOS Pathog. 2010 Dec. 2; 6(12):e1001217; Zhu and Aguzzi, J Cell Sci (2021) 134 (17): jcs245605; Fairfoul et al., 2016. Ann. Clin. Transl. Neurol. 3, 812-818; Kraus et al., 2019. Acta Neuropathol. 137, 585-598; Rossi et al., 2020. EMBO J. 20, 694-702; Saijo et al., 2017. Acta Neuropathol. 133, 751-765; Shahnawaz et al., JAMA Neurol. 2017; 74(2):163-172; and Groveman et al., Acta Neuropathol Commun. 2018; 6(1):7.

### Methods of Diagnosis and Treatment

The quantitative SAAs described herein for detecting pathological aggregates of disease-associated proteins, e.g., in plasma, serum, or plasma-derived EVs, lay the foundation for non-invasive diagnostic assays. The methods can be used for pre-symptomatic screening, enabling preventative treatment approaches to be taken earlier. A digital assay that quantifies pathological seeds provides a more robust way to classify disease severity, providing prognostic value. Additionally, quantitative methods can be used to monitor patients undergoing treatment and to evaluate the efficacy of new drugs in clinical trials. For example, a digital α-synuclein SAA can be used for detecting PD earlier and measuring changes in α-synuclein-related pathology over time. The digital SAA can be used with multiplex aggregate detection to detect multiple neurodegenerative disease-causing agents, optionally in a single sample.

The present methods can be used for any pathological neurodegenerative protein, including α-synuclein, PrP, Tau, ABeta, and TDP-43, to quantitatively diagnose multiple neurodegenerative diseases including Parkinson's disease (PD), transmissible spongiform encephalopathies (TSEs), Alzheimer's disease (AD), Alzheimer's disease (AD), amyotrophic lateral sclerosis and frontotemporal lobar degeneration, and others listed in Table A. As neurodegenerative diseases represent a growing cause of morbidity and mortality, the tests will be critical to allow for reliable prognostication and guide treatment.¹⁵

The digital SAAs described herein can be used for early screening, and in some embodiments, the presence of a level of aggregates in a sample above a reference level indicates that the subject has or is at increased risk of developing a neurodegenerative proteinopathy disease as described herein. Once such subjects are identified, the present methods can include selecting or recommending, and optionally administering, a treatment for the relevant disease (e.g., as shown in Table B), and/or suggesting and optionally conducting further testing using imaging modalities and lab tests.¹⁴ For instance, the methods can include recommending and/or sending subjects for additional testing using magnetic resonance imaging (MRI); single-photon emission computed tomography (SPECT) imaging; and/or positron emission tomography (PET). For example, using PET and SPECT imaging of presynaptic dopaminergic function, significant reductions in vesicular monoamine transporter type 2 (VMAT2), dopamine transporter (DAT), and L-aromatic amino acid decarboxylase (L-AAA) can be seen in PD patients.³⁸ Protein biomarker levels in the blood and CSF have been shown to correlate with PD symptoms.^{40,41}

TABLE B
Optional Exemplary Treatments

| Proteinopathy | Protein | |
|---|---|---|
| Disease | aggregate | Treatment |
| Parkinson's | α-synuclein | Levodopa and a peripheral decarboxylase |
| disease (PD), | | inhibitor (PDI), e.g., carbidopa; |
| Lewy Body | | Monoamine oxidase (MAO)-B inhibitors |
| Dementia (LBD) | | (e.g., selegiline, rasagiline, safinamide,; |
| and Multiple | | dopamine agonists (e.g., ropinirole, |
| System Atrophy | | pramipexole, apomorphine, amantadine, |
| (MSA) | | rotigotine); and anticholinergic agents |
| | | (e.g., trihexyphenidyl, benztropine); |
| | | acetylcholinesterase inhibitors (e.g., |
| | | rivastigmine, galantamine, donepezil); N- |
| | | methyl-D-aspartate (NMDA) agonists |
| | | (e.g., memantine); Catechol-O-methyl |
| | | transferase (COMT) inhibitors (e.g., |
| | | opicapone, entacapone, tolcapone); |
| | | Selective Serotonin Inverse Agonists |
| | | (SSIAs)(e.g., pimvanserin); adenosine |
| | | A2A receptor antagonist (e.g., |
| | | istradefylline) |
| transmissible | prion protein | PrP^{C}-lowering anti-sense |
| spongiform | (PrP) | oligonucleotides (ASOs) |
| encephalopathies | | |
| (TSEs) | | |
| Alzheimer's | Tau | Aducanumab, Cholinesterase inhibitors |
| disease (AD), | | (Donepezil, Rivastigmine, Galantamine) |
| Progressive | | Memantine |
| Supranuclear Palsy | | |
| (PSP) and Pick's | | |
| Disease (PiD) | | |
| Alzheimer's | ABeta | Aducanumab, Cholinesterase inhibitors |
| disease (AD) | | (Donepezil, Rivastigmine, Galantamine) |
| | | Memantine |
| amyotrophic lateral | transactive | Edaravone |
| sclerosis (ALS) | response DNA- | |
| and frontotemporal | binding protein | |
| lobar degeneration | 43 (TDP-43) | |
| (FTD) | | |

### EXAMPLES

The invention is further described in the following examples, which do not limit the scope of the invention described in the claims.

### Example 1. Compartmentalization in Microwells and Droplets

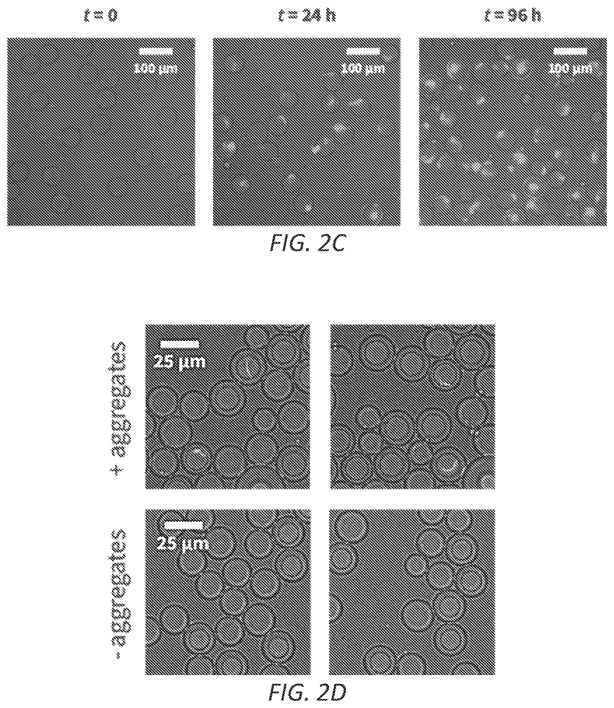

The present experiments were performed to determine whether SAAs can be carried out in microwell arrays and droplets. In these experiments, pre-formed α-synuclein fibrils were used as reaction seeds. A commercially available microfluidic device originally designed for digital PCR and shown to be point-of-care amenable¹⁶ was used (QuantStudio™, ThermoFisher). The microfluidic chips were fabricated from a silicon substrate and contained 20,000 uniformly-sized reaction wells with an approximate internal volume of 0.75 nL each. Over the course of two days, the growth of aggregates was observed when α-synuclein fibrils were spiked into the SAA reaction (FIG. 2A). If no pre-formed aggregates were added to the reaction, no fibril-like aggregates formed, and there was minimal background signal. When different concentrations of pre-formed fibrils were spiked into neurological control (NC) CSF samples, varying concentrations of fibrils were detectable down to 4 pg/mL (FIG. 2B).

In addition to microwell arrays, 20-80 ul of the SAA reaction were encapsulated in droplets. Droplets were formed using a polyethylene glycol (PEG) Krytox surfactant, where the protein-repelling PEG layer localizes to the aqueous phase.¹⁷ However, despite testing a range of buffer conditions and protein concentrations, these droplets were unstable and tended to coalesce over a short time, motivating exploration of other alternatives to create stable droplet shells. Using an ionic Krytox surfactant instead¹⁸ allowed us to bypass the issue of PEG insolubility at high salt concentrations¹⁹, significantly increasing droplet stability. We encapsulated a solution containing 5 ng/ml pre-formed aggregates, 0.1 mg/ml monomer, 100 mM PIPES pH 6.5, 100 mM GdmCl, and 1 μM X-34 dye using ionic Krytox. We imaged a fraction of the droplets at timepoints 0, 24, 96 hours. The droplets were incubated at 40° C. between the imaging steps and the droplets remained stable for multiple days. The number of droplets with aggregates and the size of the aggregates increased over time. See FIG. 2C. In a different droplets format, we used the commercially available Xdrop instrument (Samplix) to encapsulate the same solution in double emulsion droplets. The droplets were incubated at 40 C and imaged after 72 hours. Alpha-synuclein aggregates were detected in the droplets with pre-formed aggregates and were not detected in the negative control in which the reaction solution did not include aggregates. See FIG. 2D.

### Example 2. Reaction Conditions

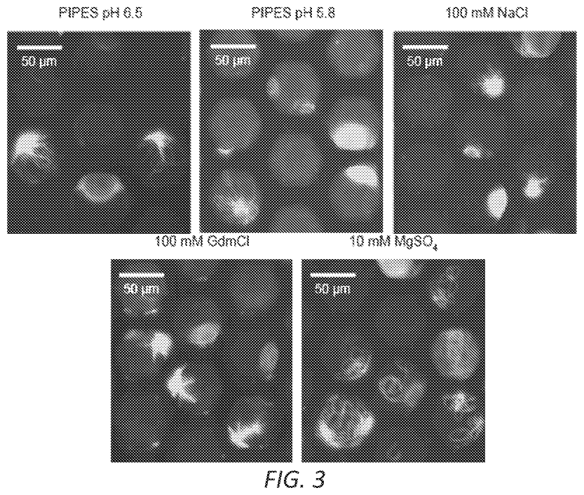

A range of SAA reaction conditions were screened to develop an optimized protocol for detecting α-synuclein fibrils in CSF samples. In vitro experiments demonstrated that the aggregation of α-synuclein is particularly sensitive to solution conditions, including temperature, pH, and salt concentration.²⁰⁻²⁴ For this reason, we investigated the effect of both pH and salt concentrations on the aggregation of α-synuclein seeds. Using pre-formed fibrils, a range of fibril morphologies was observed depending on the pH of the solution and the salt added (FIG. 3). Both 1,4-Piperazinediethanesulfonic acid (PIPES) and 4-morpholineethanesulfonic acid (MES) buffers were used to create a mildly acidic reaction environment (about pH 6.5), promoting the secondary nucleation of monomers on the fibril surface, resulting in autocatalytic amyloid amplification.²⁵ Furthermore, drawing inspiration from the Hofmeister series, ionic composition was modulated using strongly and weakly hydrated salts, such as sodium chloride (NaCl), sodium iodide (NaI), magnesium sulfate (MgSO₄), and guanidinium chloride (GdmCl). Through a systematic screening of different reaction parameters, including salts, pH, monomer concentration, and temperature, optimal reaction conditions for aggregation using CSF-derived α-synuclein seeds were determined. We tested the effect of each combination of the different components on the sensitivity and specificity of the assay. We tested serial dilution of pre-formed aggregates for sensitivity and chose the conditions in which the lowest aggregate concentration was detected; aggregates were observed in a fraction of the microwells after 3 days. We also tested the affect of pre-treatment to the working solution before the compartmentalization step. For specificity, we minimized spontaneous aggregation of the alpha-synuclein monomers without pre-formed aggregates. Optimal conditions include: 0.1 mg/ml monomer for microwells and 0.25 mg/ml for droplets, 100 mM PIPES pH 6.5, 100 mM NaCl for microwells or 100 mM GdmCl for droplets, 0.05% triton X, 0.1% BSA, and 1 μM X-34 dye, and a pre-treatment of 30 min at 40 C with continuous shaking at 400 rpm.

### Example 3. Beads

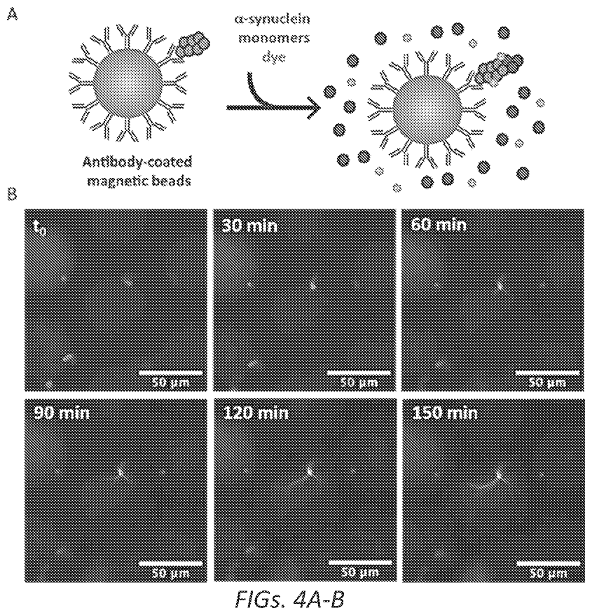

There are a number of ways to improve sensitivity. For instance, before running the SAA, we can capture α-synuclein aggregates using antibody-coated magnetic beads, where the antibody is specific for the aggregated form of α-synuclein (FIGS. 4A-B). In the first step of the assay, we mixed 100 ul of pre-formed aggregates diluted in PBS and 0.5% Triton-X, with 200,000 dye encoded magnetic beads coated with anti-alpha synuclein aggregate antibodies. The solution was incubated at room temperature for 30 min while being shaken at 800 rpm. The beads were washed with PBS three times and resuspended in the reaction solution. The optimal reaction solution included: 0.1 mg/ml monomers, 100 mM PIPES pH 6.5, 100 mM NaCl, 0.05% triton X, 0.1% BSA, 6% OptiPrep (Density Gradient Medium, Sigma), and 1 μM X-34 dye. The solution was then loaded into the microwell array and on-bead aggregate growth is monitored over the course of 1-5 days. Using this strategy with pre-formed α-synuclein fibrils, we were able to detect up to 50-fold lower concentrations. Moreover, this approach increased the specificity of the assay, since the seeds in the sample are pre-captured on the beads, any signal that is not localized to the beads can be classified as non-specific signal and can be ruled out during the off-line image analysis step.

### Example 4. Alpha-Synuclein in Plasma-Derived EVs

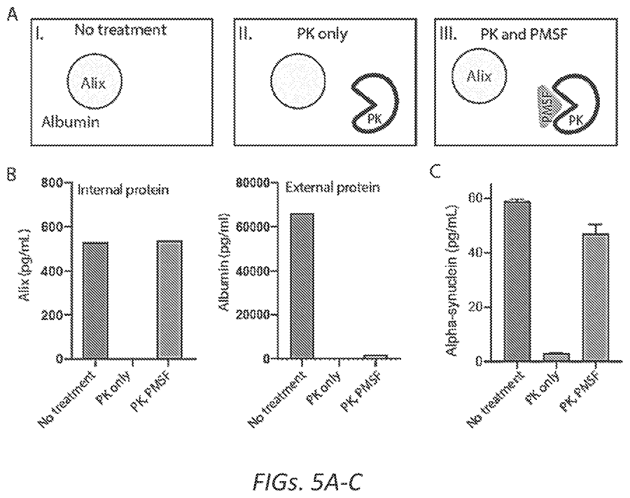

FIG. 5A provides a schematic illustration of a protection assay in which isolated EVs are treated with Proteinase K (PK) and PK inhibitor (PMSF) to quantify internal and external protein levels. As shown in FIG. 5B, to validate the protection assay, Alix (left graph) and Albumin (right graph) concentrations of EVs isolated from plasma were measured by Simoa for three conditions: i. No treatment, two-hour Project Proposal Template 5 incubation, lyse with Triton X, one-hour incubation; ii. PK treatment, two-hour incubation, lyse with Triton X, one-hour incubation; iii. PK treatment, one-hour incubation, PMSF treatment, one hour incubation, lyse with Triton X, one-hour incubation. We observed that the level of Alix was similar in conditions i and iii but disappeared completely in condition ii in which lysis occurs without PK inactivation. On the other hand, Albumin was high in condition i but close to the limit of detection in conditions ii and iii in which PK is present. These results suggest that this proteinase protection assay was suitable for quantifying protein biomarkers inside EVs. FIG. 5C shows the concentration of alpha-synuclein in EVs isolated from plasma as measured by Simoa for the three conditions, showing the presence of alpha-synuclein inside EVs.

### REFERENCES

1 Marras C, Beck J C, Bower J H, et al. Prevalence of Parkinson's disease across North America. Npj Park Dis. 2018; 4(1):1-7. doi: 10.1038/s41531-018-0058-0
2. Adler C H, Beach T G, Zhang N, et al. Unified Staging System for Lewy Body Disorders: Clinicopathologic Correlations and Comparison to Braak Staging. J Neuropathol Exp Neurol. 2019; 78(10): 891-899. doi: 10.1093/jnen/nlz080
3. Paciotti S, Bellomo G, Gatticchi L, Parnetti L. Are We Ready for Detecting α-Synuclein Prone to Aggregation in Patients? The Case of “Protein-Misfolding Cyclic Amplification” and “Real-Time Quaking-Induced Conversion” as Diagnostic Tools. Front Neurol. 2018; 0. doi: 10.3389/fneur.2018.00415
4. Saijo E, Groveman B R, Kraus A, et al. Ultrasensitive RT-QuIC Seed Amplification Assays for Disease-Associated Tau, α-Synuclein, and Prion Aggregates. In: Gomes C M, ed. Protein Misfolding Diseases: Methods and Protocols. Methods in Molecular Biology. Springer; 2019:19-37. doi: 10.1007/978-1-4939-8820-4_2
5. Fairfoul G, McGuire L I, Pal S, et al. Alpha-synuclein R T-QuIC in the CSF of patients with alpha-synucleinopathies. Ann Clin Transl Neurol. 2016; 3(10):812-818. doi: 10.1002/acn3.338
6. Shahnawaz M, Tokuda T, Waragai M, et al. Development of a Biochemical Diagnosis of Parkinson Disease by Detection of α-Synuclein Misfolded Aggregates in Cerebrospinal Fluid. JAMA Neurol. 2017; 74(2): 163-172. doi: 10.1001/jamaneurol.2016.4547
7. Groveman B R, Orrù C D, Hughson A G, et al. Rapid and ultra-sensitive quantitation of disease-associated α-synuclein seeds in brain and cerebrospinal fluid by αSyn R T-QuIC. Acta Neuropathol Commun. 2018; 6(1): 7. doi: 10.1186/s40478-018-0508-2
8. Srivastava A, Singh P K, Kumbhakar M, et al. Identifying the Bond Responsible for the Fluorescence Modulation in an Amyloid Fibril Sensor. Chem—Eur J. 2010; 16(30):9257-9263.
9. Parnetti L, Gaetani L, Eusebi P, et al. CSF and blood biomarkers for Parkinson's disease. Lancet Neurol. 2019; 18(6):573-586. doi: 10.1016/S1474-4422(19)30024-9
10. Stuendl A, Kunadt M, Kruse N, et al. Induction of α-synuclein aggregate formation by CSF exosomes from patients with Parkinson's disease and dementia with Lewy bodies. Brain. 2016; 139(2):481-494. doi: 10.1093/brain/awv346
11. Dauer W, Przedborski S. Parkinson's Disease: Mechanisms and Models. Neuron. 2003; 39(6):889-909. doi: 10.1016/S0896-6273(03)00568-3
12. Breen D P, Michell A W, Barker R A. Parkinson's disease—the continuing search for biomarkers. Clin Chem Lab Med. 2011; 49(3): 393-401. doi:10.1515/CCLM.2011.080
13. Scottish Intercollegiate Guidelines Network. Diagnosis and Pharmacological Management of Parkinson's Disease: A National Clinical Guideline. Scottish Intercollegiate Guidelines Network; 2010.
14. Erkkinen M G, Kim M O, Geschwind M D. Clinical Neurology and Epidemiology of the Major Neurodegenerative Diseases. Cold Spring Harb Perspect Biol. 2018; 10(4):a033118. doi:10.1101/cshperspect.a033118
15. Feigin V L, Nichols E, Alam T, et al. Global, regional, and national burden of neurological disorders, 1990-2016: a systematic analysis for the Global Burden of Disease Study 2016. Lancet Neurol. 2019; 18(5):459-480. doi:10.1016/S1474-4422(18)30499-X
16. Rolando J C, Jue E, Schoepp N G, Ismagilov R F. Real-Time, Digital LAMP with Commercial Microfluidic Chips Reveals the Interplay of Efficiency, Speed, and Background Amplification as a Function of Reaction Temperature and Time. Anal Chem. 2019; 91(1): 1034-1042. doi: 10.1021/acs.analchem.8b04324
17. Knowles T P J, White D A, Abate A R, et al. Observation of spatial propagation of amyloid assembly from single nuclei. Proc Natl Acad Sci. 2011; 108(36): 14746-14751. doi: 10.1073/pnas. 1105555108
18. Sukovich D J, Lance S T, Abate A R. Sequence specific sorting of DNA molecules with FACS using 3dPCR. Sci Rep. 2017; 7(1):39385. doi:10.1038/srep39385
19. Etienne G, Kessler M, Amstad E. Influence of Fluorinated Surfactant Composition on the Stability of Emulsion Drops. Macromol Chem Phys. 2017; 218(2):1600365.
20. Uversky V N, Li J, Fink A L. Evidence for a Partially Folded Intermediate in α-Synuclein Fibril Formation *. J Biol Chem. 2001; 276(14): 10737-10744. doi:10.1074/jbc.M010907200
21. Munishkina L A, Henriques J, Uversky V N, Fink A L. Role of Protein-Water Interactions and Electrostatics in α-Synuclein Fibril Formation. Biochemistry. 2004; 43(11):3289-3300. doi: 10.1021/bi034938r
22. Buell A K, Galvagnion C, Gaspar R, et al. Solution conditions determine the relative importance of nucleation and growth processes in α-synuclein aggregation. Proc Natl Acad Sci. 2014; 111(21): 7671-7676. doi:10.1073/pnas. 1315346111
23. Metrick M A, Ferreira N do C, Saijo E, et al. Million-fold sensitivity enhancement in proteopathic seed amplification assays for biospecimens by Hofmeister ion comparisons. Proc Natl Acad Sci. 2019; 116(46):23029-23039. doi: 10.1073/pnas. 1909322116
24. Pálmadóttir T, Malmendal A, Leiding T, Lund M, Linse S. Charge Regulation during Amyloid Formation of α-Synuclein. J Am Chem Soc. 2021; 143(20): 7777-7791. doi: 10.1021/jacs.1c01925
25. Gaspar R, Meisl G, Buell A K, et al. Secondary nucleation of monomers on fibril surface dominates α-synuclein aggregation and provides autocatalytic amyloid amplification. Q Rev Biophys. 2017; 50. doi:10.1017/S0033583516000172
26. Norman M, Ter-Ovanesyan D, Trieu W, et al. L1CAM is not associated with extracellular vesicles in human cerebrospinal fluid or plasma. Nat Methods. 2021; 18(6):631-634. doi: 10.1038/s41592-021-01174-8
27. Ter-Ovanesyan D, Norman M, Lazarovits R, et al. Framework for rapid comparison of extracellular vesicle isolation methods. eLife. 2021; 10:e70725. doi: 10.7554/eLife.70725
28. Rissin D M, Kan C W, Campbell T G, et al. Single-molecule enzyme-linked immunosorbent assay detects serum proteins at subfemtomolar concentrations. Nat Biotechnol. 2010; 28(6):595-599. doi: 10.1038/nbt.1641
29. Tinelli M, Kanavos P, Grimaccia F. THE VALUE OF EARLY DIAGNOSIS AND TREATMENT IN PARKINSON'S DISEASE.: 40.
30. Dorsey E R, Elbaz A, Nichols E, et al. Global, regional, and national burden of Parkinson's disease, 1990-2016: a systematic analysis for the Global Burden of Disease Study 2016. Lancet Neurol. 2018; 17(11): 939-953. doi:10.1016/S1474-4422(18)30295-3
31. Dorsey E R, Constantinescu R, Thompson J P, et al. Projected number of people with Parkinson disease in the most populous nations, 2005 through 2030. Neurology. 2007; 68(5):384-386. doi: 10.1212/01.wnl.0000247740.47667.03
32. Whetten-Goldstein K, Sloan F, Kulas E, Cutson T, Schenkman M. The Burden of Parkinson's Disease on Society, Family, and the Individual. J Am Geriatr Soc. 1997; 45(7):844-849. doi:10.1111/j.1532-5415.1997.tb01512.x
33. Hallett M. Parkinson's disease tremor: pathophysiology. Parkinsonism Relat Disord. 2012; 18:S85-S86. doi: 10.1016/S1353-8020(11)70027-X
34. Ayano G. Parkinson's Disease: A Concise Overview of Etiology, Epidemiology, Diagnosis, Comorbidity and Management. J Neurol Disord. 2016; 4(6). doi: 10.4172/2329-6895.1000298
35. Varanese S, Birnbaum Z, Rossi R, Di Rocco A. Treatment of Advanced Parkinson's Disease. Park Dis. 2011; 2010:480260. doi: 10.4061/2010/480260
36. Yang W, Hamilton J L, Kopil C, et al. Current and projected future economic burden of Parkinson's disease in the U.S. Npj Park Dis. 2020; 6(1): 1-9. doi:10.1038/s41531-020-0117-1
37. Liberman A L, Zhang H, Rostanski S K, et al. Cost-Effectiveness of Advanced Neuroimaging for Transient and Minor Neurological Events in the Emergency Department. J Am Heart Assoc. 2021; 10(12): e019001. doi: 10.1161/JAHA.120.019001
38. Politis M. Neuroimaging in Parkinson disease: from research setting to clinical practice. Nat Rev Neurol. 2014; 10(12):708-722. doi: 10.1038/nrneurol.2014.205
39. Pagano G, Niccolini F, Politis M. Imaging in Parkinson's disease. Clin Med. 2016; 16(4):371-375. doi: 10.7861/clinmedicine. 16-4-371
40. Chen-Plotkin A S, Hu W T, Siderowf A, et al. Plasma epidermal growth factor levels predict cognitive decline in Parkinson disease. Ann Neurol. 2011; 69(4):655-663. doi:10.1002/ana.22271
41. Kang J H, Irwin D J, Chen-Plotkin A S, et al. Association of Cerebrospinal Fluid β-Amyloid β-42, T-tau, P-tau181, and α-Synuclein Levels With Clinical Features of Drug-Naive Patients With Early Parkinson Disease. JAMA Neurol. 2013; 70(10): 1277-1287. doi: 10.1001/jamaneurol.2013.3861
42. Russo M J, Orru C D, Concha-Marambio L, et al. High diagnostic performance of independent alpha-synuclein seed amplification assays for detection of early Parkinson's disease. Acta Neuropathol Commun. 2021; 9(1): 179. doi:10.1186/s40478-021-01282-8
43. Mazutis L, Vasiliauskas R, Weitz D A. Microfluidic Production of Alginate Hydrogel Particles for Antibody Encapsulation and Release. Macromol Biosci. 2015; 15(12):1641-1646
44. Hatori M N, Kim S C, Abate A R. Particle-Templated Emulsification for Microfluidics-Free Digital Biology. Anal Chem. 2018; 90(16):9813-9820.
45. Yelleswarapu, V.; Buser, J. R.; Haber, M.; Baron, J.; Inapuri, E.; Issadore, D., Mobile platform for rapid sub-picogram-per-milliliter, multiplexed, digital droplet detection of proteins. Proceedings of the National Academy of Sciences 2019, 116 (10), 4489.
46. Cohen, L.; Walt, D. R., Highly Sensitive and Multiplexed Protein Measurements. Chemical Reviews 2019, 119 (1), 293-321.
47. Rissin, D. M.; Walt, D. R., Digital Readout of Target Binding with Attomole Detection Limits via Enzyme Amplification in Femtoliter Arrays. Journal of the American Chemical Society 2006, 128 (19), 6286-6287.
48. Rissin, D. M.; Walt, D. R., Digital Concentration Readout of Single Enzyme Molecules Using Femtoliter Arrays and Poisson Statistics. Nano Letters 2006, 6 (3), 520-523.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for detecting, and optionally quantifying a level of,
pathological protein aggregates in a sample from a subject, preferably wherein the sample comprises cerebrospinal fluid, plasma, or serum, the method comprising:
providing a sample from a subject, optionally a subject who is known or suspected to have a condition associated with aggregation of disease-associated proteins;
optionally enriching the sample for extracellular vesicles;
diluting a portion of the sample by addition of a diluent comprising reaction reagents to form a reaction mixture, wherein the reaction reagents comprise monomers of disease-associated proteins and one or more detection reagents;
compartmentalizing the reaction mixture into separate confined volumes, preferably wherein each of the confined volumes has a volume of 1 pL to 1000 nL and wherein each confined volume has zero or one pathological protein aggregate;
incubating the confined volumes under conditions to allow co-aggregation of the monomers of disease-associated protein and any pathological protein aggregates present in the sample;
detecting the presence or absence of co-aggregates of the disease-associated protein monomers and pathological protein aggregates in each confined volume, wherein the formation of co-aggregates indicates the presence of pathological protein aggregates in the confined volume; and
optionally quantifying the level of pathological aggregates in the sample by counting the number of confined volumes comprising co-aggregates.

2. The method of claim 1, wherein the sample is diluted to a 1:1 to 1:100 ratio of sample:diluent.

3. The method of claim 1, wherein compartmentalizing the reaction mixture into separate confined volumes comprises partitioning aliquots of the reaction mixture into droplets, microwells, or hydrogel microspheres.

4. The method of claim 1, further comprising heating the confined volumes to 35-50° C. prior to detecting the presence or absence of co-aggregates.

5. The method of claim 1, further comprising mixing the sample with beads prior to compartmentalization and incubation.

6. The method of claim 5, wherein the beads are coated with antibodies that bind specifically to the co-aggregates.

7. The method of claim 1, wherein detecting the presence or absence of co-aggregates comprises detecting the presence or absence of a detection reagent signal.

8. The method of claim 1, wherein the detection reagent is Thioflavin T (ThT) or X-34, and detecting the presence or absence of a protein aggregate comprises detecting a fluorescence signal from the detection reagent.

9. The method of claim 1, wherein the sample comprises cerebrospinal fluid, plasma, or serum.

10. The method of claim 1, wherein the subject is known or suspected to have a condition associated with aggregation of disease-associated proteins, and wherein the condition associated with aggregation of pathological proteins is Parkinson's disease (PD), and the pathological protein monomers comprise α-synuclein; the condition associated with aggregation of pathological proteins is transmissible spongiform encephalopathies (TSEs) and the pathological protein monomers comprise prion protein (PrP); the condition associated with aggregation of pathological proteins is Alzheimer's disease (AD), and the pathological protein monomers comprise Amyloid Beta; the condition associated with aggregation of pathological proteins is amyotrophic lateral sclerosis (ALS) and frontotemporal lobar degeneration (FTD), and the pathological protein monomers comprise transactive response DNA-binding protein 43 (TDP-43); or the condition associated with aggregation of pathological proteins is Pick disease, Alzheimer's disease (AD), chronic traumatic encephalopathy (CTE), progressive supranuclear palsy (PSP), corticobasal degeneration (CBD), frontotemporal dementias associated with MAPT mutations (FTDP 17 MAPT), and the pathological protein monomers comprise Tau.

11. (canceled)

12. (canceled)

13. (canceled)

14. (canceled)

15. A method of detecting, and optionally quantifying a level of,
pathological protein aggregates of two or more different disease-associated proteins in a sample from a subject, preferably wherein the sample comprises cerebrospinal fluid, plasma, or serum, the method comprising:
providing a sample from a subject, optionally a subject who is known or suspected to have a condition associated with aggregation of disease-associated proteins;
optionally enriching the sample for extracellular vesicles;
mixing the sample with a plurality of fluorescently labeled beads, wherein the beads are coated with antibodies that bind specifically to each of the two or more co-aggregates, preferably α-synuclein, PrP, amyloid beta, TDP-43, and/or tau co-aggregates, wherein each fluorescent label is associated with antibodies that bind specifically to a specific protein aggregate;
diluting the mixture of sample and beads by addition of a diluent comprising reaction reagents to form a reaction mixture, wherein the reaction reagents comprise monomers of disease-associated proteins and a detection reagent;
compartmentalizing the reaction mixture into separate confined volumes, preferably wherein each of the confined volumes has a volume of 1 pL to 1000 nL, and wherein each confined volume has zero or one pathological protein aggregate;
incubating the confined volumes under conditions to allow co-aggregation of the monomers of disease-associated protein and any pathological protein aggregates present in the sample;
detecting the presence or absence of co-aggregates of the disease-associated protein monomers and pathological protein aggregates in each confined volume, wherein the formation of co-aggregates indicates the presence of pathological protein aggregates in the confined volume;
determining the identity of the co-aggregates based on the identity of fluorescent labels, and optionally quantifying the level of each of the pathological protein aggregates in the sample by counting the number of confined volumes comprising co-aggregates.

16. The method of claim 15, wherein the sample is diluted to a 1:1 to 1:100 ratio of sample:diluent.

17. The method of claim 15, wherein compartmentalizing the reaction mixture into separate confined volumes comprises partitioning aliquots of the reaction mixture into droplets, microwells, or hydrogel microspheres.

18. The method of claim 15, further comprising heating the confined volumes to 35-50° C. prior to detecting the presence or absence of co-aggregates.

19. The method of claim 15, wherein detecting the presence or absence of co-aggregates comprises detecting the presence or absence of a detection reagent signal.

20. The method of claim 15, wherein the detection reagent is Thioflavin T (ThT) or X-34, and detecting the presence or absence of a protein aggregate comprises detecting a fluorescence signal from the detection reagent.

21. The method of claim 15, wherein the sample comprises cerebrospinal fluid, plasma, or serum.

22. The method of claim 15, further comprising identifying the subject as having a condition associated with aggregation of pathological proteins

23. The method of claim 15, wherein the two or more disease-associated proteins are selected from the group consisting of α-synuclein, PrP, amyloid beta, TDP-43, and tau.

24. The method of claim 22, wherein the condition associated with aggregation of pathological proteins is Parkinson's disease (PD), and the pathological protein monomers comprise α-synuclein; the condition associated with aggregation of pathological proteins is transmissible spongiform encephalopathies (TSEs) and the pathological protein monomers comprise prion protein (PrP); the condition associated with aggregation of pathological proteins is Alzheimer's disease (AD), and the pathological protein monomers comprise Amyloid Beta; the condition associated with aggregation of pathological proteins is amyotrophic lateral sclerosis (ALS) and frontotemporal lobar degeneration (FTD), and the pathological protein monomers comprise transactive response DNA-binding protein 43 (TDP-43); or the condition associated with aggregation of pathological proteins is Pick disease, Alzheimer's disease (AD), chronic traumatic encephalopathy (CTE), progressive supranuclear palsy (PSP), corticobasal degeneration (CBD), frontotemporal dementias associated with MAPT mutations (FTDP 17 MAPT), and the pathological protein monomers comprise Tau.

25.-28. (canceled)
